(19) United States
(12) Patent Application Publication
(10) Pub. No.: US 2022/0356545 A1
(43) Pub. Date: Nov. 10, 2022

(54) LITHIUM EXTRACTANT COMPOUNDS AND THEIR USE IN SELECTIVE LITHIUM EXTRACTION FROM AQUEOUS SOLUTIONS

(71) Applicant: UT-Battelle, LLC, Oak Ridge, TN (US)

(72) Inventors: Ilja Popovs, Knoxville, TN (US); Henry Musrock, East Lansing, MI (US); Katherine R. Johnson, Knoxville, TN (US); Bruce A. Moyer, Oak Ridge, TN (US)

(21) Appl. No.: 17/726,890

(22) Filed: Apr. 22, 2022

Related U.S. Application Data
(60) Provisional application No. 63/216,114, filed on Jun. 29, 2021.
(60) Provisional application No. 63/178,613, filed on Apr. 23, 2021.

(51) Int. Cl. C22B 26/12; C01D 15/00; C07C 235/74; C22B 3/34; C22B 3/36

(52) U.S. Cl. CPC C22B 26/12; C01D 15/00; C07C 235/74; C22B 3/34; C22B 3/36

(57) ABSTRACT

Lithium extractant compounds having the following structure:

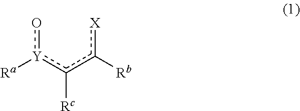

wherein: R^{a} and R^{b} are independently selected from the group consisting of hydrocarbon groups (R), —OR, —NRR′, —SR, —SO₂R, —SO₂NR₂, —C(O)R, —C(O)OR, —C(O)NRR′, —C(S)OR, —C(O)SR, and —C(S)NRR′; R′ is selected from R′ groups, wherein R′ is selected from H and R groups; X is O or OH; Y is C or N, wherein, when Y is N, then R^{a} is R. Also described are hydrophobic water-insoluble solutions containing at least one extractant compound of Formula (1). Also described is a method for extracting lithium from an aqueous solution by contacting the aqueous solution with the hydrophobic solution, and optional stripping of lithium from the hydrophobic solution by contacting the hydrophobic solution with an aqueous stripping solution.

### CROSS REFERENCE TO RELATED APPLICATION

The present application claims benefit of U.S. Provisional Application No. 63/178,613, filed on Apr. 23, 2021, and U.S. Provisional Application No. 63/216,114, filed on Jun. 29, 2021, all of the contents of which are incorporated herein by reference.

### STATEMENT REGARDING FEDERALLY SPONSORED RESEARCH

This invention was made with government support under Prime Contract No. DE-AC05-00OR22725 awarded by the U.S. Department of Energy. The government has certain rights in the invention.

### FIELD OF THE INVENTION

The present invention generally relates to materials and methods for extracting lithium from aqueous solutions. The present invention more particularly relates to lithium complexing compounds and their use in extracting lithium from aqueous solutions.

### BACKGROUND OF THE INVENTION

Methods for the selective extraction and concentration of lithium from terrestrial brines, geothermal brines, and other lithium-containing solutions containing other cations, such as sodium (Na⁺), and potassium (K⁺), at high concentrations are continually being sought but with little success. The conventional methods are generally energy intensive and time consuming. For example, thermal or solar evaporation generally requires heating by combustion of fossil fuels or reliance on solar radiation and wind, any of which typically requires 18-24 months to produce a final lithium salt product. Moreover, water consumption and water management have become issues of significant concern, particularly since a large number of evaporation ponds and lithium production sites are located in arid regions of the world, such as the Atacama dessert.

As lithium has gained importance as an element for use in various applications, there are continuing efforts to develop less costly and more efficient methods for the recovery of lithium. In particular, there have been significant efforts in the use of layered lithium aluminates, typically of the formula LiX/Al(OH)₃, such as described in, for example, U.S. Pat. Nos. 9,012,357, 8,901,032, 8,753,594, 8,637,428, 6,280,693, 4,348,295, and 4,461,714. Unfortunately, such methods, which generally employ packed columns for the recovery, suffer from a number of drawbacks, such as shortened lifetimes due to the gradual deterioration and disintegration of the particles and collapse of the crystal structures. Lithium-manganese oxide compositions have also been used, but they tend to suffer from instability from the use of concentrated acid to recover lithium from the sorbent. The use of packed columns, normally even with the best designs of liquid distribution, cannot prevent mixing of loading and regeneration streams, which results in contamination of even the most selective of sorbent materials.

There would thus be a significant advantage in a process that could bypass the shortcomings of the conventional art and directly extract lithium in a selective manner from aqueous solutions containing high concentrations of other ions. Such a process, which has thus far remained elusive, would provide a more cost-effective and economical alternative to extracting lithium than the currently available technology.

### SUMMARY OF THE INVENTION

In a first aspect, the present disclosure is directed to novel compounds having an exceptional ability in selectively chelating lithium ions and transporting them into a substantially hydrophobic phase. The lithium extractant compounds possess precisely or at least two coordinating functional groups, typically selected from carbonyl, hydroxy, amine oxide, and combinations thereof and at least one or two hydrocarbon groups containing 1-30 carbon atoms to confer a hydrophobic property.

More specifically, the lithium extractant compounds have the following structure:

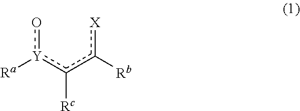

In Formula (1), R^{a} and R^{b} are independently selected from hydrocarbon groups (R), —OR, —NRR′, —SR, —SO₂R, —SO₂NR₂, —C(O)R, —C(O)OR, —C(O)NRR′, —C(S)OR, —C(O)SR, and —C(S)NRR′; R′ is selected from R′ groups; wherein the hydrocarbon groups (R) independently contain 1-30 carbon atoms and are selected from alkyl groups, alkenyl groups, alkynyl groups, cycloalkyl groups, cycloalkenyl groups, aromatic groups, and heteroaromatic groups, any of which are optionally substituted with fluorine, and wherein said cycloalkyl groups, cycloalkenyl groups, aromatic groups, and heteroaromatic groups are further optionally substituted with one or more groups selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂; R′ is selected from H and R groups; X is O or OH; Y is C or N, wherein, when Y is N, then R^{a} is R; the dotted lines indicate optional presence of carbon-carbon double bonds; R^{a} and R^{c} optionally interconnect via their R groups to form ring A, wherein ring A is optionally substituted with one or more groups selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂; R^{b} and R′ optionally interconnect via their R groups to form ring B, wherein ring B is optionally substituted with one or more groups selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂; and R^{a}, R^{b}, and R^{c} optionally interconnect via their R groups to form a fused bicyclic ring system, wherein the fused bicyclic ring system is optionally substituted with one or more groups selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂; provided that R^{a} and R^{b} are not both alkyl groups, and if R^{a} is an alkyl group and R^{b} is an aromatic group, or if R^{a} and R^{b} are the same aromatic groups, then at least one of the aromatic groups is substituted with a group selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂.

In a second aspect, the present disclosure is directed to an aqueous-insoluble hydrophobic solution (i.e., “liquid solution”) useful for extracting lithium from an aqueous solution. The liquid solution contains at least one lithium extractant compound according to Formula (1) dissolved in an aqueous-insoluble (hydrophobic) solvent. The aqueous-insoluble hydrophobic solvent may be, for example, a hydrocarbon solvent. In some embodiments, the liquid solution further contains a non-chelating co-extractant molecule dissolved in the aqueous-insoluble hydrophobic solvent, wherein the non-chelating co-extractant molecule has a single donor group that can form a single coordinate bond with a lithium ion. The non-chelating co-extractant molecule may be selected from, for example, R′C(O)NR′₂, R′₂NC(O)NR′₂, C(O)R₂, P(O)R₃, S(O)R₂, and N⁺(O⁻)R′₃, wherein: R is independently selected from hydrocarbon group containing 1-30 carbon atoms and selected from alkyl groups, alkenyl groups, alkynyl groups, cycloalkyl groups, cycloalkenyl groups, and aromatic groups, with no heteroatom substitution; R′ is selected from H and R; and one or more R groups in P(O)R₃ and C(O)R₂ can be replaced with an OR group.

In a third aspect, the present disclosure is directed to a method for extracting lithium from an aqueous solution. The method includes the following steps, at minimum: (i) providing an aqueous solution containing lithium and having a pH sufficient to deprotonate a chelating lithium extractant compound according to Formula (1); (ii) contacting the aqueous solution with an aqueous-insoluble hydrophobic solution, as described above, containing the chelating lithium extractant compound dissolved in an aqueous-insoluble hydrophobic solvent. Step (ii) results in deprotonation of the chelating lithium extractant compound along with simultaneous chelation of lithium in the alkalized aqueous solution by the deprotonated form of the chelating lithium extractant compound, and resultant extraction of the lithium from the aqueous solution into the aqueous-insoluble hydrophobic solution.

The lithium extraction process described herein is advantageously straight-forward and cost-efficient while at the same time capable of removing a substantial portion or all of the lithium from an aqueous source, and further capable of achieving this with high selectivity for lithium even while in the presence of higher concentrations of other metal ions, such as sodium and potassium. The process also advantageously does not rely on thermal or solar evaporation, nor does the process rely on sorbent methods, such as columns packed with layered lithium aluminate sorbents. Thus, the process described herein circumvents the significant drawbacks and problems associated with the conventional methods, and instead relies on a straight-forward, cost-effective, and quick method for directly and selectively removing lithium from lithium-containing solutions.

### DETAILED DESCRIPTION OF THE INVENTION

As used herein, the term “hydrocarbon group” (also denoted by the group R) is defined as a chemical group composed of at least of carbon and hydrogen. In some embodiments, the hydrocarbon group is composed solely of carbon and hydrogen, except that the hydrocarbon group may (i.e., optionally) be substituted with one or more fluorine atoms to result in partial or complete fluorination of the hydrocarbon group. In other embodiments, as further discussed below, the hydrocarbon group may be substituted with one or more heteroatom-containing groups.

The hydrocarbon group typically contains 1-30 carbon atoms. In different embodiments, one or more of the hydrocarbon groups may contain, for example, 1, 2, 3, 4, 5, 6, 7, 8, 9, 10, 12, 15, 18, 20, 22, 24, 26, 28, or 30 carbon atoms, or a number of carbon atoms within a particular range bounded by any two of the foregoing carbon numbers (e.g., 1-30, 2-30, 3-30, 4-30, 6-30, 8-30, 10-30, 12-30, 1-20, 6-20, 8-20, 10-20, or 12-20 carbon atoms). Hydrocarbon groups in different compounds described herein, or in different positions of a compound, may possess the same or different number (or preferred range thereof) of carbon atoms in order to independently adjust or optimize such properties as the complexing ability, extracting (extraction affinity) ability, or selectivity ability.

In a first set of embodiments, the hydrocarbon group (R) is a saturated and straight-chained group, i.e., a straight-chained (linear) alkyl group. Some examples of straight-chained alkyl groups include methyl, ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-eicosyl, n-docosyl, n-tetracosyl, n-hexacosyl, n-octacosyl, and n-triacontyl groups.

In a second set of embodiments, the hydrocarbon group (R) is saturated and branched, i.e., a branched alkyl group. Some examples of branched alkyl groups include isopropyl (2-propyl), isobutyl (2-methylprop-1-yl), sec-butyl (2-butyl), t-butyl (1,1-dimethylethyl-1-yl), 2-pentyl, 3-pentyl, 2-methylbut-1-yl, isopentyl (3-methylbut-1-yl), 1,2-dimethylprop-1-yl, 1,1-dimethylprop-1-yl, neopentyl (2,2-dimethylprop-1-yl), 2-hexyl, 3-hexyl, 2-methylpent-1-yl, 3-methylpent-1-yl, isohexyl (4-methylpent-1-yl), 1,1-dimethylbut-1-yl, 1,2-dimethylbut-1-yl, 2,2-dimethylbut-1-yl, 2,3-dimethylbut-1-yl, 3,3-dimethylbut-1-yl, 1,1,2-trimethylprop-1-yl, 1,2,2-trimethylprop-1-yl, isoheptyl, isooctyl, and the numerous other branched alkyl groups having up to 20 or 30 carbon atoms, wherein the “1-yl” suffix represents the point of attachment of the group.

In a third set of embodiments, the hydrocarbon group (R) is saturated and cyclic, i.e., a cycloalkyl group. Some examples of cycloalkyl groups include cyclopropyl, cyclobutyl, cyclopentyl, cyclohexyl, cycloheptyl, and cyclooctyl groups. The cycloalkyl group can also be a polycyclic (e.g., bicyclic) group by either possessing a bond between two ring groups (e.g., dicyclohexyl) or a shared (i.e., fused) side (e.g., decalin and norbornane).

In a fourth set of embodiments, the hydrocarbon group (R) is unsaturated and straight-chained, i.e., a straight-chained (linear) olefinic or alkenyl group. The unsaturation occurs by the presence of one or more carbon-carbon double bonds and/or one or more carbon-carbon triple bonds. Some examples of straight-chained olefinic groups include vinyl, propen-1-yl (allyl), 3-buten-1-yl (CH₂═CH—CH₂—CH₂—), 2-buten-1-yl (CH₂—CH═CH—CH₂—), butadienyl, 4-penten-1-yl, 3-penten-1-yl, 2-penten-1-yl, 2,4-pentadien-1-yl, 5-hexen-1-yl, 4-hexen-1-yl, 3-hexen-1-yl, 3,5-hexadien-1-yl, 1,3,5-hexatrien-1-yl, 6-hepten-1-yl, ethynyl, propargyl (2-propynyl), 3-butynyl, and the numerous other straight-chained alkenyl or alkynyl groups having up to 20 or 30 carbon atoms.

In a fifth set of embodiments, the hydrocarbon group (R) is unsaturated and branched, i.e., a branched olefinic or alkenyl group. Some examples of branched olefinic groups include propen-2-yl (CH₂═C.—CH₃), 1-buten-2-yl (CH₂═C.—CH₂—CH₃), 1-buten-3-yl (CH₂═CH—CH.—CH₃), 1-propen-2-methyl-3-yl (CH₂═C(CH₃)—CH₂—), 1-penten-4-yl, 1-penten-3-yl, 1-penten-2-yl, 2-penten-2-yl, 2-penten-3-yl, 2-penten-4-yl, and 1,4-pentadien-3-yl, and the numerous other branched alkenyl groups having up to 20 or 30 carbon atoms, wherein the dot in any of the foregoing groups indicates a point of attachment.

In a sixth set of embodiments, the hydrocarbon group (R) is unsaturated and cyclic, i.e., a cycloalkenyl group. The unsaturated cyclic group may be aromatic or aliphatic. Some examples of unsaturated cyclic hydrocarbon groups include cyclopropenyl, cyclobutenyl, cyclopentenyl, cyclopentadienyl, cyclohexenyl, cyclohexadienyl, phenyl, benzyl, cycloheptenyl, cycloheptadienyl, cyclooctenyl, cyclooctadienyl, and cyclooctatetraenyl groups. The unsaturated cyclic hydrocarbon group may or may not also be a polycyclic group (such as a bicyclic or tricyclic polyaromatic group) by either possessing a bond between two of the ring groups (e.g., biphenyl) or a shared (i.e., fused) side, as in naphthalene, anthracene, phenanthrene, phenalene, or indene fused ring systems.

As indicated earlier above, any of the hydrocarbon groups described above may be substituted with one or more fluorine atoms. As an example, an n-octyl group may be substituted with a single fluorine atom to result in, for example, a 7-fluorooctyl or 8-fluorooctyl group, or substituted with two or more fluorine atoms to result in, for example, 7,8-difluorooctyl, 8,8-difluorooctyl, 8,8,8-trifluorooctyl, or perfluorooctyl group. As also indicated earlier above, any of the hydrocarbon groups described above may contain a single ether (—O—) or thioether (—S—) linkage connecting between carbon atoms in the hydrocarbon group. An example of a hydrocarbon group containing a single ether or thioether group is —(CH₂)₂—X—(CH₂)₇CH₃, wherein X represents O or S.

As further indicated earlier above, any of the hydrocarbon groups described above may be substituted with one or more heteroatom-containing groups. Some examples of heteroatom-containing groups include —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂ groups, wherein R″ groups are independently selected from alkyl groups containing 1-20 carbon atoms.

In one aspect, the present disclosure is directed to lithium extractant compounds having an ability to complex with lithium ions and transfer (extract) the lithium from an aqueous solution into an aqueous-insoluble hydrophobic (non-polar) solution in which the extractant compound is dissolved. The extractant compound possesses precisely or at least two coordinating functional groups, typically selected from carbonyl, hydroxy, amine oxide, and combinations thereof and at least one or two hydrocarbon groups containing 1-30 carbon atoms to confer a hydrophobic property. The term “compound” is herein meant to be synonymous with the term “molecule”.

The lithium extractant compounds are within the following generic structure:

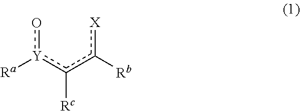

In Formula (1) above, R^{a} and R^{b} are independently selected from hydrocarbon groups (R), —OR, —NRR′, —SR, —SO₂R, —SO₂NR₂, —C(O)R, —C(O)OR, —C(O)NRR′, —C(S)OR, —C(O)SR, and —C(S)NRR′, wherein R has been described above, and R′ is selected from H and R groups. R′ is selected from R′ groups. In different embodiments, the total carbon atoms in R^{a}, R^{b}, and R^{c} is at least 12, 13, 14, 15, 16, 18, 20, 22, 24, 26, 28, 30, 32, 34, 36, 38, 40, 42, 44, 46, 48, 50, 52, 54, 56, 60, 64, 68, 70, 72, 76, or 80, or a total carbon number within a range bounded by any two of the foregoing values (e.g., 12-80). In some embodiments, at least one or both of R^{a} and R^{b} is/are independently selected from —C(O)R, —C(O)OR, —C(O)NRR′, —C(S)OR, —C(O)SR, —C(S)NRR′, and —NRR′. The variable X is O or OH; the variable Y is C or N, wherein, when Y is N, then R^{a} is R, which may or may not be substituted with one, two, or more heteroatom-containing groups described earlier above. The dotted lines indicate optional presence of carbon-carbon double bonds. The structure according to Formula (1) may be symmetric or asymmetric.

Typically, the hydrocarbon groups (R) in Formula (1) are selected from alkyl groups, alkenyl groups, alkynyl groups, cycloalkyl groups, cycloalkenyl groups, aromatic groups, and heteroaromatic groups, any of which are optionally substituted with fluorine, and wherein the cycloalkyl groups, cycloalkenyl groups, aromatic groups, and heteroaromatic groups. Any one or more of the foregoing groups may be optionally substituted with one or more groups selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂ groups. In some embodiments, at least one, two, or all of R^{a}, R^{b}, and R^{c} are selected from hydrocarbon groups (R), or any one or more of the particular types of R groups provided above (e.g., aromatic or heteroatomic groups), wherein R in any one or more of R^{a}, R^{b}, and R^{c} is/are substituted by at least one of —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂ groups, or substituted by at least one of —C(O)R, —C(O)OR, —C(O)NRR′, —C(S)OR, —C(O)SR, and —C(S)NRR′.

In some embodiments of Formula (1), R^{a} and R^{c} optionally interconnect via their R groups to form ring A, wherein ring A is optionally substituted with one or more groups selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. Ring A may be saturated or unsaturated (or aromatic) and typically contains five, six, or seven ring atoms, which may be all carbon atoms or carbon atoms and one or more heteroatoms (typically nitrogen or oxygen). In some embodiments, ring A is a benzene ring.

In some embodiments of Formula (1), R^{b} and R^{c} optionally interconnect via their R groups to form ring B, wherein ring B is optionally substituted with one or more groups selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. Ring B may be saturated or unsaturated (or aromatic) and typically contains five, six, or seven ring atoms, which may be all carbon atoms or carbon atoms and one or more heteroatoms (typically nitrogen or oxygen). In some embodiments, ring B is a benzene ring.

In yet other embodiments of Formula (1), R^{a}, R^{b}, and R^{c} optionally interconnect via their R groups to form a fused bicyclic ring system, wherein the fused bicyclic ring system is optionally substituted with one or more groups selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. Rings A and/or B may be saturated or unsaturated (or aromatic) and typically contains five, six, or seven ring atoms, which may be all carbon atoms or carbon atoms and one or more heteroatoms (typically nitrogen or oxygen). In some embodiments, ring A and/or B is/are benzene ring(s).

Compounds according to Formula (1) with ring A, ring B, or rings A and B are generically depicted as follows:

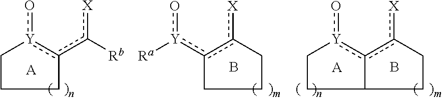

wherein n and m are independently 1, 2, or 3, which correspond to five-membered, six-membered, and seven-membered rings, respectively.

Notably, for purposes of the present invention, compounds of Formula (1) having R^{a} and R^{b} as both alkyl groups (i.e., with no substitution with heteroatoms or heteroatom-containing groups) are not included. Moreover, if R^{a} is an alkyl group and R^{b} is an aromatic group, or if R^{a} and R^{b} are the same aromatic groups, then at least one of the aromatic groups is substituted with a group selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂.

In some embodiments of Formula (1), X is O and R^{b} is a phenyl ring, which results in the lithium extractant compound having the following structure:

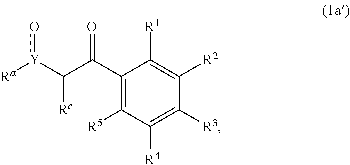

or more particularly, the following structure when Y is C and R^{c} is hydrogen:

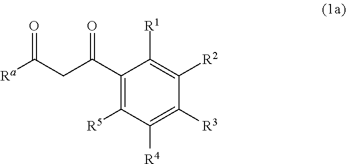

In Formula (1a′) or (1a), R^{a} and R^{c} are as defined above, including any of the specific embodiments provided, including the possibility of R^{a} and R^{c} interconnecting to form a ring, and/or R¹ and R⁵ interconnecting to form a ring, and R¹, R², R³, R⁴, and R⁵ are independently selected from H, alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. The rings may be defined as rings A and/or B, as defined earlier above. In some embodiments, precisely or at least one of R¹, R², R³, R⁴, and R⁵ is selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In other embodiments, precisely or at least two or three of R¹, R², R³, R⁴, and R⁵ are selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In any of the foregoing groups, R″ may independently be any of the alkyl groups provided earlier above. In some embodiments, precisely or at least one (or two or three) of R¹, R², R³, R⁴, and R⁵ is/are —OR″ or —OH groups. In some embodiments of Formula (1a′) or (1a), R^{a} is a linear or branched alkyl or alkenyl group containing 2-30, 3-30, 4-30, 5-30, 6-30, 7-30, or 8-30 carbon atoms.

In other embodiments of Formula (1), X is O and R^{a} and R^{b} are both phenyl rings, which results in the lithium extractant compound having the following structure:

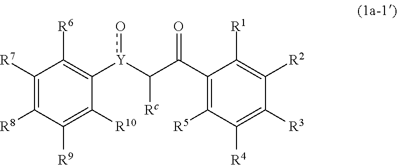

or more particularly, the following structure when Y is C and R^{c} is hydrogen:

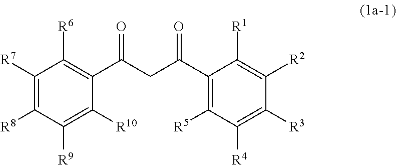

In Formula (1a-1′) or (1a-1), R′ is as defined above, including any of the specific embodiments provided, including the possibility of R^{a} and R^{c} interconnecting to form a ring, and/or R^{c} and R⁵ or R¹⁰ interconnecting to form a ring, and R¹, R², R³, R⁴, R⁵, R⁶, R⁷, R⁸, R⁹, and R¹⁰ are independently selected from H, alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. The rings may be defined as rings A and/or B, as defined earlier above. In some embodiments, precisely or at least one of R¹, R², R³, R⁴, and R⁵ and/or precisely or at least one of R⁶, R⁷, R⁸, R⁹, and R¹⁰ is selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In other embodiments, precisely or at least two or three of R¹, R², R³, R⁴, and R⁵ and/or precisely or at least two or three of R⁶, R⁷, R⁸, R⁹, and R¹⁰ are selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In any of the foregoing groups, R″ may independently be any of the alkyl groups provided earlier above. In some embodiments, precisely or at least one (or two or three) of R¹, R², R³, R⁴, and R⁵ and/or precisely or at least one (or two or three) of R⁶, R⁷, R⁸, R⁹, and R¹⁰ is/are —OR″ or —OH groups.

In other embodiments of Formula (1), X is O and R^{b} is an amino (—NRR′) group, which results in the lithium extractant compound having the following structure:

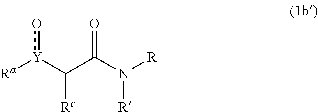

or more particularly, the following structure when Y is C and R^{c} is hydrogen:

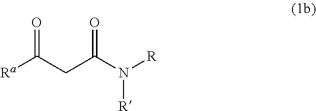

In Formula (1b′) or (1b), R^{a} and R^{c} are as defined above, including any of the specific embodiments provided, including the possibility of R^{a} and R^{c} interconnecting to form a ring, and/or R^{c} and R′ interconnecting to form a ring, or both. The rings may be defined as rings A and/or B, as defined earlier above. As defined earlier above, the group R is a hydrocarbon group containing 1-30 carbon atoms, and R′ is selected from H and R. In some embodiments, R^{a} is or includes a ring, particularly an aromatic group (e.g., benzene ring) which may (optionally) be substituted with one or more groups selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In other embodiments, R^{a} is or includes a ring (e.g., benzene ring) which may be substituted with precisely or at least two or three groups selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In any of the foregoing groups, R″ may independently be any of the alkyl groups provided earlier above. In some embodiments, R^{a} is or includes a ring (e.g., benzene ring) which may be substituted with precisely or at least one (or two or three) —OR″ and/or —OH groups. In some embodiments of Formula (1b′) or (1b), R^{a} is a linear or branched alkyl or alkenyl group containing 2-30, 3-30, 4-30, 5-30, 6-30, 7-30, or 8-30 carbon atoms.

In other embodiments of Formula (1), X is O and R^{b} is an alkoxy (—OR) group, which results in the lithium extractant compound having the following structure:

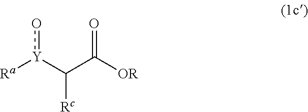

or more particularly, the following structure when Y is C and R^{c} is hydrogen:

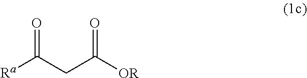

In Formula (1c′) or (1c), R^{a} and R^{c} are as defined above, including any of the specific embodiments provided, including the possibility of R^{a} and R^{c} interconnecting to form a ring, and/or R^{c} and R interconnecting to form a ring, or both. The rings may be defined as rings A and/or B, as defined earlier above. As defined earlier above, the group R is a hydrocarbon group containing 1-30 carbon atoms. In some embodiments, R^{a} is or includes a ring, particularly an aromatic group (e.g., benzene ring) which may (optionally) be substituted with one or more groups selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In other embodiments, R^{a} is or includes a ring (e.g., benzene ring) which may be substituted with precisely or at least two or three groups selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In any of the foregoing groups, R″ may independently be any of the alkyl groups provided earlier above. In some embodiments, R^{a} is or includes a ring (e.g., benzene ring) which may be substituted with precisely or at least one (or two or three) —OR″ and/or —OH groups. In some embodiments of Formula (1c′) or (1c), R^{a} is a linear or branched alkyl or alkenyl group containing 2-30, 3-30, 4-30, 5-30, 6-30, 7-30, or 8-30 carbon atoms.

In other embodiments of Formula (1), X is OH and R^{b} and R^{c} are interconnected to form a benzene ring, which results in the lithium extractant compound having the following structure:

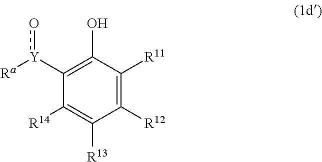

or more particularly, the following structure when Y is C:

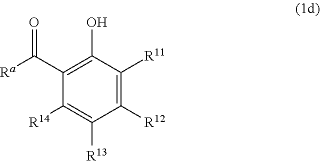

In Formula (1d′) or (1d), R^{a} is as defined above, including any of the specific embodiments provided, including the possibility of R^{a} and R¹⁴ interconnecting to form a ring fused to the benzene ring, and R¹¹, R¹², R¹³, and R¹⁴ are independently selected from H, alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. The ring fused to the benzene ring, if present, may be defined as ring A or B, as defined earlier above, including the possibility of the fused ring containing a ring heteroatom, such as an O or N atom. In some embodiments, precisely or at least one of R¹¹, R¹², R¹³, and R¹⁴ is selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In other embodiments, precisely or at least two or three of R¹¹, R¹², R¹³, and R¹⁴ are selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In any of the foregoing groups, R″ may independently be any of the alkyl groups provided earlier above. In some embodiments, precisely or at least one (or two or three) of R¹¹, R¹², R¹³, and R¹⁴ is/are —OR″ or —OH groups. In some embodiments, R^{a} is or includes a ring, particularly an aromatic group (e.g., benzene ring) which may (optionally) be substituted with one or more groups selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In other embodiments, R^{a} is or includes a ring (e.g., benzene ring) which may be substituted with precisely or at least two or three groups selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In any of the foregoing groups, R″ may independently be any of the alkyl groups provided earlier above. In some embodiments, R^{a} is or includes a ring (e.g., benzene ring) which may be substituted with precisely or at least one (or two or three) groups selected from R″, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂ groups, ore more particularly, one or more —OR″ and/or —OH groups. In some embodiments of Formula (1d′) or (1d), R^{a} is a linear or branched alkyl or alkenyl group containing 2-30, 3-30, 4-30, 5-30, 6-30, 7-30, or 8-30 carbon atoms. In other specific embodiments, R^{a} may be —OR or —NRR′.

In other embodiments of Formula (1), X is OH and R^{a}, R^{b}, and R^{c} are interconnected to form a fused ring system, or more particularly, a naphthyl ring system, in which case the lithium extractant compound may have the following structure:

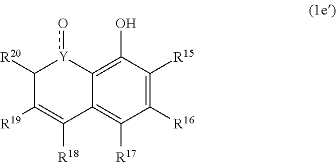

or more particularly, the following structure when Y is C:

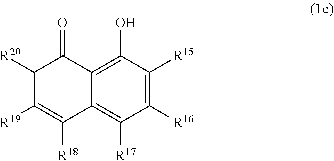

In Formula (1e′) or (1e), R¹⁵, R¹⁶, R¹⁷, R¹⁸, R¹⁹, and R²⁰ are independently selected from H, alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. Typically, at least one of R¹⁵, R¹⁶, R¹⁷, R¹⁸, R¹⁹, and R²⁰ is selected from R″, OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂, or at least one of R¹⁵, R¹⁶, R¹⁷, R¹⁸, R¹⁹, and R²⁰ is selected from R″, —OR″, —NHR″, —NR″₂, —SR″, —C(O)R″, —C(O)OR″, —C(O)NHR″, —C(O)NR″₂, —C(S)OR″, —C(O)SR″, —C(S)NHR″, and —C(S)NR″₂. In some embodiments, precisely or at least one of R¹⁵, R¹⁶, and R¹⁷ and/or precisely or at least one of R¹⁸, R¹⁹, and R²⁰ is selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In other embodiments, precisely or at least two or three of R¹⁵, R¹⁶, and R¹⁷ and/or precisely or at least two or three of R¹⁸, R¹⁹, and R²⁰ are selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In any of the foregoing groups, R″ may independently be any of the alkyl groups provided earlier above. In some embodiments, precisely or at least one (or two or three) of R¹⁵, R¹⁶, and R¹⁷ and/or precisely or at least one (or two or three) of R¹⁸, R¹⁹, and R²⁰ is/are —OR″ or —OH groups.

In other embodiments of Formula (1), X is OH and R^{a}, R^{b}, and R^{c} are interconnected to form a fused ring system, or more particularly, a naphthyl ring system, with at least one ring atom in the fused ring system (or naphthyl ring system) being an uncharged nitrogen atom. The lithium extractant compounds may, for example, have a ring nitrogen atom adjacent to a ring carbonyl group, in which case the lithium extract compound may have the following structure:

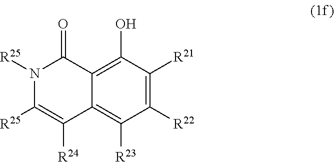

In Formula (1f), R²¹, R²², R²³, R²⁴, and R²⁵ are independently selected from H, alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂, and R²⁶ is selected from R″, —NHR″, —NR″₂, —NO₂, —C(O)R″, —C(O)OR″, —C(O)NHR″, —C(O)NR″₂, —C(S)OR″, —C(O)SR″, —C(S)NHR″, and —C(S)NR″₂. In some embodiments, at least one of R²¹, R²², R²³, R²⁴, and R²⁵ is selected from R″, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In some embodiments, precisely or at least one of R²¹, R²², and R²³ and/or precisely or at least one of R²⁴ and R²⁵ is selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In other embodiments, precisely or at least two or three of R²¹, R²², and R²³ and/or precisely or one or both of R²⁴ and R²⁵ are selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In any of the foregoing groups, R″ may independently be any of the alkyl groups provided earlier above.

In some embodiments, precisely or at least one (or two or three) of R²¹, R²², and R²³ and/or precisely or at least one of R²⁴ and R²⁵ is/are —OR″ or —OH groups.

In other embodiments of Formula (1), X is OH, Y is N, and R^{a}, R^{b}, and R^{c} are interconnected to form a fused ring system, or more particularly, a naphthyl ring system, in which case the lithium extractant compound may have the following structure:

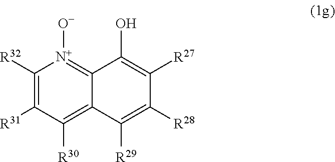

In Formula (1g), R²⁷, R²⁸, R²⁹, R³⁰, R³¹, and R³² are independently selected from H, alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In some embodiments, at least one of R¹⁵, R¹⁶, R¹⁷, R¹⁸, R¹⁹, and R²⁰ is selected from R″, OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂, or at least one of R²⁷, R²⁸, R²⁹, R³⁰, R³¹, and R³² is selected from R″, —OR″, —NHR″, —NR″₂, —SR″, —C(O)R″, —C(O)OR″, —C(O)NHR″, —C(O)NR″₂, —C(S)OR″, —C(O)SR″, —C(S)NHR″, and —C(S)NR″₂. In some embodiments, precisely or at least one of R²⁷, R²⁸, and R²⁹ and/or precisely or at least one of R³⁰, R³¹, and R³² is selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In other embodiments, precisely or at least two or three of R²⁷, R²⁸, and R²⁹ and/or precisely or at least two or three of R³⁰, R³¹, and R³² are selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In any of the foregoing groups, R″ may independently be any of the alkyl groups provided earlier above. In some embodiments, precisely or at least one (or two or three) of R²⁷, R²⁸, and R²⁹ and/or precisely or at least one (or two or three) of R³⁰, R³¹, and R³² is/are —OR″ or —OH groups.

In some embodiments, at least one or both of R^{a} and R^{b} is —C(O)R, in which case the lithium extract compound may have any of the following structures:

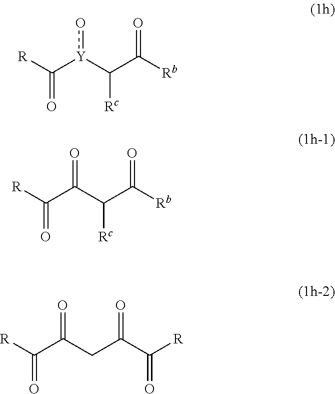

In Formula (1h), (1h-1), and (1h-2), R^{b} and R^{c} are as defined above, including any of the specific embodiments provided, including the possibility of R^{b} and R^{c} interconnecting to form a ring. The ring may be defined as ring A or B, as defined earlier above. As defined earlier above, the group R is independently a hydrocarbon group containing 1-30 carbon atoms. In some embodiments, R^{b} is or includes a ring, particularly an aromatic group (e.g., benzene ring) which may (optionally) be substituted with one or more groups selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In other embodiments, R^{b} is or includes a ring (e.g., benzene ring) which may be substituted with precisely or at least two or three groups selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In any of the foregoing groups, R″ may independently be any of the alkyl groups provided earlier above. In some embodiments, R^{b} is or includes a ring (e.g., benzene ring) which may be substituted with precisely or at least one (or two or three) —OR″ and/or —OH groups. In some embodiments of Formula (1h) or (1h-1), R^{b} is a linear or branched alkyl or alkenyl group containing 2-30, 3-30, 4-30, 5-30, 6-30, 7-30, or 8-30 carbon atoms.

In some embodiments, at least one or both of R^{a} and R^{b} is —C(O)OR, in which case the lithium extract compound may have any of the following structures:

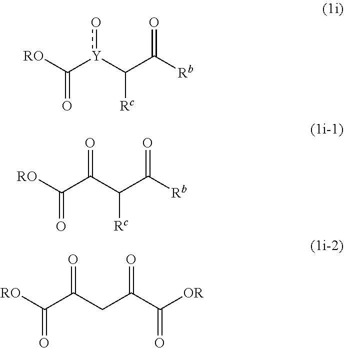

In Formula (1i), (1i-1), and (1i-2), R^{b} and R^{c} are as defined above, including any of the specific embodiments provided, including the possibility of R^{b} and R^{c} interconnecting to form a ring. The ring may be defined as ring A or B, as defined earlier above. As defined earlier above, the group R is a hydrocarbon group containing 1-30 carbon atoms. In some embodiments, R^{b} is or includes a ring, particularly an aromatic group (e.g., benzene ring) which may (optionally) be substituted with one or more groups selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In other embodiments, R^{b} is or includes a ring (e.g., benzene ring) which may be substituted with precisely or at least two or three groups selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In any of the foregoing groups, R″ may independently be any of the alkyl groups provided earlier above. In some embodiments, R^{b} is or includes a ring (e.g., benzene ring) which may be substituted with precisely or at least one (or two or three) —OR″ and/or —OH groups. In some embodiments of Formula (1h) or (1h-1), R^{b} is a linear or branched alkyl or alkenyl group containing 2-30, 3-30, 4-30, 5-30, 6-30, 7-30, or 8-30 carbon atoms.

In some embodiments, at least one or both of R^{a} and R^{b} is —C(O)NR′₂, in which case the lithium extract compound may have any of the following structures:

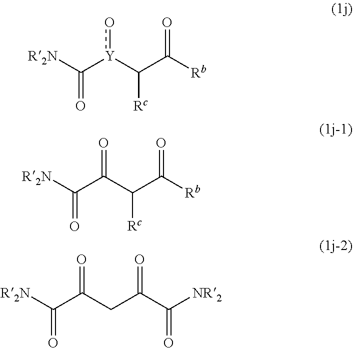

In Formula (1j), (1j-1), and (1j-2), R^{b} and R^{c} are as defined above, including any of the specific embodiments provided, including the possibility of R^{b} and R^{c} interconnecting to form a ring. The ring may be defined as ring A or B, as defined earlier above. As defined earlier above, the group R′ is independently selected from H and R groups, wherein R is a hydrocarbon group containing 1-30 carbon atoms. In some embodiments, R′ does not include H. In some embodiments, R^{b} is or includes a ring, particularly an aromatic group (e.g., benzene ring) which may (optionally) be substituted with one or more groups selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In other embodiments, R^{b} is or includes a ring (e.g., benzene ring) which may be substituted with precisely or at least two or three groups selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In any of the foregoing groups, R″ may independently be any of the alkyl groups provided earlier above. In some embodiments, R^{b} is or includes a ring (e.g., benzene ring) which may be substituted with precisely or at least one (or two or three) —OR″ and/or —OH groups. In some embodiments of Formula (1j), (1j-1), or (1j-2), R^{b} is a linear or branched alkyl or alkenyl group containing 2-30, 3-30, 4-30, 5-30, 6-30, 7-30, or 8-30 carbon atoms.

In some embodiments, R^{a} is —C(O)NR′₂ and R^{b} is —C(O)R, in which case the lithium extract compound may have any of the following structures:

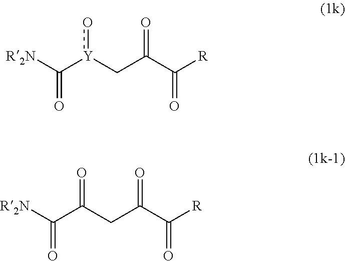

In Formula (1k) and (1k-1), the group R′ is independently selected from H and R groups, wherein R is independently a hydrocarbon group containing 1-30 carbon atoms, as described above. In some embodiments, R′ does not include H. In some embodiments, R shown in any of the above formulas is or includes a ring, particularly an aromatic group (e.g., benzene ring) which may (optionally) be substituted with one or more groups selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In other embodiments, R shown in any of the above formulas is or includes a ring (e.g., benzene ring) which may be substituted with precisely or at least two or three groups selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In any of the foregoing groups, R″ may independently be any of the alkyl groups provided earlier above. In some embodiments, R shown in any of the above formulas is or includes a ring (e.g., benzene ring) which may be substituted with precisely or at least one (or two or three) —OR″ and/or —OH groups. In some embodiments of Formula (1k) or (1k-1), R shown in any of the above formulas is a linear or branched alkyl or alkenyl group containing 2-30, 3-30, 4-30, 5-30, 6-30, 7-30, or 8-30 carbon atoms.

In some embodiments, R^{a} is —C(O)NR′₂ and R^{b} is —C(O)OR, in which case the lithium extract compound may have any of the following structures:

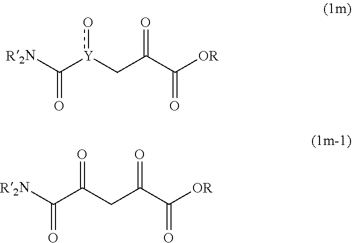

In Formula (1m) and (1m-1), the group R′ is independently selected from H and R groups, wherein R is independently a hydrocarbon group containing 1-30 carbon atoms, as described above. In some embodiments, R′ does not include H. In some embodiments, R shown in any of the above formulas is or includes a ring, particularly an aromatic group (e.g., benzene ring) which may (optionally) be substituted with one or more groups selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In other embodiments, R shown in any of the above formulas is or includes a ring (e.g., benzene ring) which may be substituted with precisely or at least two or three groups selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In any of the foregoing groups, R″ may independently be any of the alkyl groups provided earlier above. In some embodiments, R shown in any of the above formulas is or includes a ring (e.g., benzene ring) which may be substituted with precisely or at least one (or two or three) —OR″ and/or —OH groups. In some embodiments of Formula (1m) or (1m-1), R shown in any of the above formulas is a linear or branched alkyl or alkenyl group containing 2-30, 3-30, 4-30, 5-30, 6-30, 7-30, or 8-30 carbon atoms.

In some embodiments, R^{a} is —C(O)NR′₂ and R^{b} is —NR′₂, in which case the lithium extract compound may have any of the following structures:

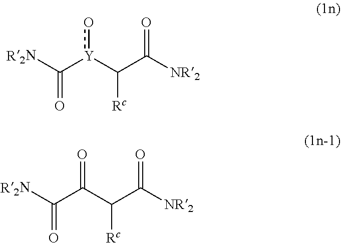

In Formula (1n) and (1n-1), R′ and R^{c} are as defined above, including any of the specific embodiments provided, including the possibility of any one of R′ interconnecting with R^{c} to form a ring. The ring may be defined as ring A or B, as defined earlier above. The group R′ is independently selected from H and R groups, wherein R is independently a hydrocarbon group containing 1-30 carbon atoms, as described above. In some embodiments, R′ does not include H. In some embodiments, precisely or at least one or two R′ groups is or includes a ring, particularly an aromatic group (e.g., benzene ring) which may (optionally) be substituted with one or more groups selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In other embodiments, precisely or at least one or two R′ groups is or includes a ring (e.g., benzene ring) which may be substituted with precisely or at least two or three groups selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In any of the foregoing groups, R″ may independently be any of the alkyl groups provided earlier above. In some embodiments, precisely or at least one or two R groups is or includes a ring (e.g., benzene ring) which may be substituted with precisely or at least one (or two or three) —OR″ and/or —OH groups. In some embodiments of Formula (1n) or (in-1), precisely or at least one or two R′ groups is a linear or branched alkyl or alkenyl group containing 2-30, 3-30, 4-30, 5-30, 6-30, 7-30, or 8-30 carbon atoms.

In some embodiments, R^{a} and R^{b} are both —NR′₂, in which case the lithium extract compound may have the following structure:

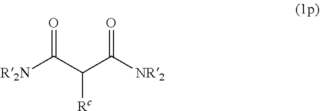

In Formula (1p), R′ and R^{c} are as defined above, including any of the specific embodiments provided, including the possibility of any one or two of R′ interconnecting with R^{c} to form a ring or fused ring system. The ring may independently be defined as ring A or B, as defined earlier above, and a fused ring system may include any combination of rings A and B fused together. The group R′ is independently selected from H and R groups, wherein R is independently a hydrocarbon group containing 1-30 carbon atoms, as described above. In some embodiments, R′ does not include H. In some embodiments, precisely or at least one or two R′ groups is or includes a ring, particularly an aromatic group (e.g., benzene ring) which may (optionally) be substituted with one or more groups selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In other embodiments, precisely or at least one or two R′ groups is or includes a ring (e.g., benzene ring) which may be substituted with precisely or at least two or three groups selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In any of the foregoing groups, R″ may independently be any of the alkyl groups provided earlier above. In some embodiments, precisely or at least one or two R′ groups is or includes a ring (e.g., benzene ring) which may be substituted with precisely or at least one (or two or three) —OR″ and/or —OH groups. In some embodiments of Formula (1p), precisely or at least one or two R′ groups is a linear or branched alkyl or alkenyl group containing 2-30, 3-30, 4-30, 5-30, 6-30, 7-30, or 8-30 carbon atoms.

In some embodiments, R^{a} and R^{b} are both —OR, in which case the lithium extract compound may have the following structure:

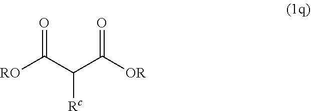

In Formula (1q), the group R′ is as defined above. The group R is independently a hydrocarbon group containing 1-30 carbon atoms, as described above. Formula (1q) includes the possibility of any one or two of R interconnecting with R′ to form a ring or fused ring system. The ring may independently be defined as ring A or B, as defined earlier above, and a fused ring system may include any combination of rings A and B fused together. In some embodiments, one or both R groups is or includes a ring, particularly an aromatic group (e.g., benzene ring) which may (optionally) be substituted with one or more groups selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In other embodiments, one or both R groups is or includes a ring (e.g., benzene ring) which may be substituted with precisely or at least two or three groups selected from alkyl groups (R″) containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂. In any of the foregoing groups, R″ may independently be any of the alkyl groups provided earlier above. In some embodiments, one or both R groups is or includes a ring (e.g., benzene ring) which may be substituted with precisely or at least one (or two or three) —OR″ and/or —OH groups. In some embodiments of Formula (1q), one or both R groups is a linear or branched alkyl or alkenyl group containing 2-30, 3-30, 4-30, 5-30, 6-30, 7-30, or 8-30 carbon atoms.

The lithium extractant compounds according any of the formulas provided above can be synthesized by methods well known in the art. In typical embodiments, compounds that are the derivatives of salicylic acid can be obtained by amidation or esterification of the corresponding acid. Benzophenone- and acetophenone-based extractants can be prepared by a well-known Friedel-Crafts acylation reactions of substituted phenols with corresponding activated benzoic acids. The derivatives of heterocycle-N-oxides can be prepared from the corresponding parent heterocycle using oxygen transfer reagents, such as peroxyacids, peroxides and their various derivatives. The derivatives of 3-oxopropanoic acid can be conveniently prepared by reaction of amine or alcohol with the functionalized diketene derivative under catalyzed or thermal conditions. The derivatives of 2,4-dioxobutanoic, 2,4-dioxopentanoic acids and their derivatives can be conveniently obtained through a condensation reaction of methyl-ketones, such as acetophenone or pinacolone, or acetamides, or acetate esters with oxalic acid monoamide-monoester or diester derivatives under basic conditions similar to literature procedures (Jian Wang, et al. Synthesis and first X-ray structures of cobalt(II) and cobalt(III) complexes bearing 2,4-dioxo-alkanoic acid dialkylamide ligands. Canadian Journal of Chemistry. 87(1): 328-334. https://doi.org/10.1139/v08-151).

Preparation of N,N-bis(2-ethylhexyl)-3-oxobutanamide (5). A mixture of 2,2,6-trimethyl-4H-1,3-dioxin-4-one (2.84 g, 20 mmol) and N,N-bis(2-ethylhexyl)amine was heated to 90° C. and stirred overnight. The residue was purified by silica gel column chromatography (silica gel, petroleum ether/EtOAc, v/v 5:1). N,N-bis(2-ethylhexyl)-3-oxobutanamide (5) (90% yield) as a yellow oil.

The Examples, provided later below, describe a number of methods for producing these compounds.

Some specific examples of lithium extractant compounds include: N,N-bis(2-ethylhexyl)-3-oxobutanamide, 2-ethylhexyl salicylate, N,N-bis(2-ethylhexyl)-2,4-dioxo-4-phenylbutanamide, N,N-bis(2-ethylhexyl)-3-oxodecanamide, N,N-bis(2-ethylhexyl)-3-oxo-3-(2,4,6-trichlorophenyl)propanamide, N,N-bis(2-ethylhexyl)-3-oxo-3-phenylpropanamide, N,N-bis(2-ethylhexyl)-4,4-dimethyl-3-oxopentanamide, N,N-bis(2-ethylhexyl)-2,4-dioxopentanamide, N,N-bis(2-ethylhexyl)-3-oxodecanamide, N,N-bis(2-ethylhexyl)-3-(4-methoxyphenyl)-3-oxopropanamide, N,N-bis(2-ethylhexyl)-2,4-dioxopentanamide, N,N-bis(2-ethylhexyl)-3-oxodecanamide, N,N-bis(2-ethylhexyl)-3-(4-methoxyphenyl)-3-oxopropanamide, N,N-bis(2-ethylhexyl)-5,5-dimethyl-2,4-dioxohexanamide, 2-ethylhexyl 2,4-dioxopentanoate, 2-ethylhexyl 2,4-dioxo-4-phenylbutanoate, 2-ethylhexyl 5,5-dimethyl-2,4-dioxohexanoate, N,N-bis(2-ethylhexyl)-2,4,5-trioxo-5-phenylpentanamide, octyl 2,4-dioxopentanoate, N,N-bis(2-ethylhexyl)-2,4,5-trioxo-5-phenylpentanamide, N,N-bis(2-ethylhexyl)-6,6-dimethyl-2,4,5-trioxoheptanamide, N,N-bis(2-ethylhexyl)-2,4,5-trioxohexanamide, (4-(bis(2-ethylhexyl)amino)phenyl)(2-hydroxy-5-nitrophenyl)methanone, 5,7-dibutyl-8-hydroxyquinoline 1-oxide, 3,7-dibutyl-8-hydroxyquinoline 1-oxide, 3,7-dibutyl-8-hydroxy-5-nitroquinoline 1-oxide, (2-hydroxy-5-nitrophenyl)(4-pentylphenyl)methanone, (5-hexadecyl-2-hydroxyphenyl)(4-pentylphenyl)methanone, 8-hydroxyquinoline 1-oxide, 8-hydroxy-2-methyl-5-nitroisoquinolin-1(2H)-one, 8-hydroxy-5-nitro-2-(4-propyltridecyl)isoquinolin-1(2H)-one, 2-hexadecyl-8-hydroxy-6-(5-pentyldodecyl)isoquinolin-1(2H)-one, octyl 5-(bis(2-ethylhexyl)amino)-2,4,5-trioxopentanoate, N,N-bis(2-ethylhexyl)-2,4,5-trioxo-5-phenylpentanamide, (4-(dimethylamino)phenyl)(2-hydroxyphenyl)methanone, 7-hydroxy-5-(2-octyldecyl)phenanthridin-6(5H)-one, N,N-bis(2-ethylhexyl)-2-hydroxybenzamide, 10-(tert-butylsulfonyl)-7-hydroxy-5-octylphenanthridin-6(5H)-one, N1,N1-bis(2-ethylhexyl)-N5-octyl-2,4-dioxo-N5-phenylpentanediamide, (4-(bis(2-ethylhexyl)amino)phenyl)(5-hexadecyl-2-hydroxyphenyl)methanone, (5-chloro-2-hydroxyphenyl)(4-(dioctylamino)phenyl)methanone, 5-(2-butylhexyl)-10-fluoro-7-hydroxyphenanthridin-6(5H)-one, 2-(tert-butyl)-8-chloro-5-(2-ethylhexyl)-7-hydroxyphenanthridin-6(5H)-one, 2-hydroxy-5-nitro-N,N-dioctylbenzamide, 3,5-dichloro-2-hydroxy-N,N-dioctylbenzamide, 3,5-dichloro-N,N-bis(2-ethylhexyl)-2-hydroxybenzamide, and (4-(dimethylamino)phenyl)(5-hexadecyl-2-hydroxyphenyl)methanone.

In another aspect, the present disclosure is directed to a liquid extractant solution useful for extracting lithium from aqueous solutions. The liquid extractant solution is aqueous-insoluble. The extraction solution includes one or more extractant compounds (i.e., any one or more of the lithium extractant compounds of Formula (1) or any sub-formula thereof or species thereof, described above), dissolved in an aqueous-insoluble hydrophobic solvent. The aqueous-insoluble hydrophobic solvent can be any of the hydrophobic organic solvents known in the art that are substantially or completely immiscible with water or aqueous solutions in general. The aqueous-insoluble hydrophobic solvent is typically a hydrocarbon solvent, which may be non-halogenated (e.g., hexanes, heptanes, octanes, decanes, dodecanes, benzene, toluene, xylenes, kerosene, or petroleum ether), or halogenated (e.g., methylene chloride, chloroform, carbon tetrachloride, 1,2-dichlorethane, trichloroethylene, and perchloroethylene), or etherified (e.g., diethyl ether or diisopropyl ether), or combination of halogenated and etherified (e.g., bis(chloroethyl)ether and 2-chloroethyl vinyl ether). In some embodiments, the extractant solution is composed solely of the extractant compound and the aqueous-insoluble hydrophobic solvent. The one or more extractant compounds may be present in the extractant solution in a concentration of, for example, precisely, at least, or up to, for example, 0.01 M, 0.02 M, 0.05 M, 0.1 M, 0.2 M, 0.25 M, 0.3 M, 0.4 M, 0.5 M, 0.6 M, 0.7 M, 0.8 M, 0.9 M, or 1 M or a concentration within a range bounded by any two of the foregoing values, e.g., 0.01-1 M, 0.01-0.5 M, 0.01-0.3 M, 0.01-0.25 M, 0.05-1 M, 0.05-0.5 M, 0.05-0.3 M, 0.05-0.25 M, 0.1-1 M, 0.1-0.8 M, 0.1-0.5 M, 0.1-0.25 M, 0.15-1 M, 0.15-0.8 M, 0.15-0.5 M, 0.15-0.3 M, 0.15-0.25 M, 0.2-1 M, 0.2-0.8 M, or 0.2-0.5 M.

In some embodiments, the aqueous-insoluble hydrophobic solution further includes a non-chelating co-extractant molecule dissolved in the aqueous-insoluble hydrophobic solvent, wherein the non-chelating co-extractant molecule has a single donor group that can form a single coordinate bond with a lithium ion. In some embodiments, the extractant solution is composed solely of the extractant compound, co-extractant molecule(s), and the aqueous-insoluble hydrophobic solvent. The non-chelating co-extractant molecule may be one or more molecules selected from, for example, R′C(O)NR′₂, R′₂NC(O)NR′₂, C(O)R₂, P(O)R₃, S(O)R₂, and N⁺(O⁻)R′₃, wherein R is independently selected from hydrocarbon groups containing 1-30 carbon atoms and selected from alkyl groups, alkenyl groups, alkynyl groups, cycloalkyl groups, cycloalkenyl groups, and aromatic groups, with no heteroatom substitution, and R′ is selected from H and R. In some embodiments, the co-extractant molecule contains at least or above two, three, four, five, six, seven, eight, nine, ten, eleven, or twelve carbon atoms. The co-extractant molecule may also be in cyclic form, such as by interconnecting any two of the R′ and/or R groups in the molecule. Some examples of R′C(O)NR′₂ co-extractant molecules include dimethylformamide, diethylformamide, dimethylacetamide, and diethylacetamide. Some examples of R′₂NC(O)NR′₂ co-extractant molecules include tetramethylurea, 1,3-diethylurea, tetraethylurea, and tetrabutylurea. Some examples of C(O)R₂ co-extractant molecules include 3-pentanone, 2-hexanone, and 3-hexanone. Some examples of P(O)R₃ co-extractant molecules include triethylphosphine oxide, tibutylphosphine oxide, trihexylphosphine oxide, and trioctylphosphine oxide (TOPO). In some embodiments, the one or more R groups in P(O)R₃ and C(O)R₂ can be replaced with an OR group, such as in P(O)(OR)₃ and RC(O)OR co-extracant molecules. Some examples of S(O)R₂ co-extractant molecules include dimethyl sulfoxide, diethyl sulfoxide, and dibutyl sulfoxide. Some examples of N⁺(O⁻)R′₃ co-extractant molecules include trimethylamine oxide, triethylamine oxide, tripropylamine oxide, tributylamine oxide, and trioctylamine oxide. Any one or more of the foregoing classes or species of co-extractant molecules may be excluded from the hydrophobic solution. The co-extractant can be independently included in any of the amounts provided above for the extractant molecule.

In some embodiments, the extractant solution contains one or more additional components, such as one or more phase modifiers. Phase modifiers may be, for example, hydrocarbon chain alcohols, including but not limited to, for example, 1-octanol, 1-isooctanol, 1-decanol, 1-dodecanol, or more generally, isomeric branched or linear, primary alcohols that contain both even- and odd-numbered hydrocarbon chains, ranging from C₁ to C₃₀ and/or a mixture of any number of these alcohols or their derivatives including but not limited to esters, ethers, organophosphates, carbonates, and the like. In some embodiments, one or more (or all) such phase modifiers or any additional components is/are excluded from the extractant solution.

In another aspect, the present disclosure is directed to a method for extracting lithium from an aqueous source solution containing lithium. In a first step of the extraction process (i.e., step (i)), the aqueous solution containing lithium is first obtained (i.e., provided). The pH of the aqueous solution should be sufficiently high to deprotonate the lithium extractant compound according to any of Formula (1) or sub-formulas thereof. Since the lithium extractant compounds vary in their acidity, the pH necessary to effect deprotonation can also widely vary. Thus, for more acidic lithium extractant compounds, a pH of 5, 6, 7, or 8 may be sufficient to result in deprotonation, and conversely, for less acidic lithium extractant compounds, a pH of 8, 9, 10, 11, 12, or 13 may be more suitable. Depending on the lithium extractant compound(s) being used in the hydrophobic extracting solution, the pH of the aqueous solution may be within a range of any of the foregoing values (e.g., 5-14, 5-13, 6-14, 6-13, 7-14, 7-13, 8-14, 8-13, 9-14, or 9-13). In the case where the aqueous solution is brine, the pH is typically high, such as a pH of 14, and thus, a pH adjuster is generally not included. However, in cases where the aqueous solution is less alkaline, the pH of the aqueous solution can be raised by addition of a base, such as an alkali hydroxide (e.g., NaOH or KOH) or an amine (e.g., ammonia or trimethylamine).

In a second step of the extraction process (i.e., step (ii)), the aqueous solution from step (i) is contacted with the above-described aqueous-insoluble hydrophobic extracting solution containing an extractant compound of Formula (1) and optionally further including one or more co-extractant molecules, such as any one or more of those described above. Any of the concentrations provided above for the extractant compound and co-extractant molecule may be used in the method. The term “contacted” or “contacting,” as used herein in reference to contacting of the aqueous and organic (hydrophobic) phases, generally refers to an intimate mixing of the aqueous and organic phases so as to maximize extraction of the lithium from the aqueous phase to the organic phase. In the extraction process, the lithium extractant compound in the hydrophobic (organic) phase is deprotonated upon contact with the aqueous solution, and the resulting deprotonated extractant compound readily and selectively chelates to lithium ions in the aqueous phase and transports (extracts) them into the organic phase. Methods of intimately mixing liquids are well known in the art. For example, the aqueous and organic phases may be placed in a container and the container agitated. In some embodiments, the liquids are intimately mixed by subjecting them to vortex mixing. Following mixing, the two phases are generally separated by means well known in the art, such as by standing or centrifugation. The foregoing described process amounts to an efficient liquid-liquid extraction process whereby lithium in the aqueous source solution is selectively extracted into the aqueous-insoluble hydrophobic solvent (organic phase).

The aqueous and organic phases may be used in any suitable volume ratio, wherein the volume of the organic phase is referred to as V_{org} and the volume of the aqueous phase is referred to as V_{aq}. In different embodiments, the V_{org}:V_{aq} (O:A) ratio is precisely or at least, for example, 0.5:1, 0.75:1, 1:1, 1.25:1, 1.5:1, 1.75:1, 2:1, 2.25:1, 2.5:1, 2.75:1, 3:1, 3.25:1, 3.5:1, 3.75:1, or 4:1, or a ratio within a range bounded by any two of the foregoing ratios, e.g., 0.5:1-4:1, 1:1-4:1, 0.5-3:1, or 1:1-3:1.

Once the lithium is extracted into the organic phase, the lithium is typically removed (stripped) from the organic phase in order to isolate it in the form of a usable salt. The lithium may be stripped from the aqueous-insoluble hydrophobic solution by contacting the aqueous-insoluble hydrophobic solution with an aqueous stripping solution having a pH sufficient to result in reprotonation of the chelating lithium extractant compound and simultaneous release of the lithium from the aqueous-insoluble hydrophobic solution into the aqueous stripping solution. The pH needed for reprotonation of the extractant compound is dependent on the acidity of the extractant compound. In cases where the extractant compound is more acidic, the pH used for reprotonation may be, for example, 1, 2, 3, 4, 5, 6, or 7. In cases where the extractant compound is less acidic, the pH used for reprotonation may be, for example, 7, 8, or 9. If needed, to suitably lower the pH, the aqueous stripping solution may contain an inorganic acid or organic acid, any of which may be a strong or weak acid. Some examples of inorganic acids include hydrohalides (i.e., HX, wherein X is typically Cl, Br, or I), sulfuric acid (H₂SO₄), nitric acid (HNO₃), and phosphoric acid (H₃PO₄). Some examples of organic acids include carboxylic acids (e.g., acetic or propionic acid) and sulfonic acids (e.g., triflic acid). In some embodiments, one or more of the foregoing classes or species of acids is excluded from the aqueous stripping solution.

The extraction process is generally capable of achieving a distribution coefficient (D), which may also herein be referred to as an extraction affinity, of at least 1 for lithium, wherein D is the concentration ratio of lithium in the organic phase divided by its concentration in the aqueous phase. In some embodiments, a D value of greater than 1 is achieved, such as a D value of at least or above 2, 5, 10, 20, 50, 100, 150, 200, 250, 500, or 1000. The selectivity of the process can be characterized by the separation factor (SF), wherein SF is calculated as the ratio of D for lithium over the D of any other ion in the aqueous source solution. Selectivity is generally evident in an SF value greater than 1. In some embodiments, an SF value of at least or greater than 2, 5, 10, 20, 50, 100, 150, 200, 250, 500, or 1000 is achieved.

In some embodiments, the aqueous solution contains lithium along with at least one other type of ion, such as sodium, potassium, magnesium, and/or calcium. In such cases, the extraction step (step ii) generally extracts lithium to a greater degree (i.e., by a greater D value) than one or more other types of ions. By extracting lithium to a greater degree than one or more other elements, the extraction step is exhibiting a degree in selectivity for lithium. The degree of selectivity can be adjusted by, for example, selection of the lithium extracting compound according to Formula (1); selection of the concentration of the lithium extracting compound; selection of the co-extractant molecule, if present; and selection of the volume ratio of organic and aqueous phases.

Examples have been set forth below for the purpose of illustration and to describe certain specific embodiments of the invention. However, the scope of this invention is not to be in any way limited by the examples set forth herein.

### Examples

Reagents and Materials

Isopar L, dibenzoylmethane (1), butylmethoxydibenzoylmethane (2), 2-ethylhexyl salicylate (3), 2,2,6,6-tetramethyl-3,5-heptanedione (dipivaloylmethane) (4), trioctylphosphine oxide (TOPO), and Cyanex® 923 (mixture of trialkylphosphine oxides) were commercially obtained and used without further purification. Compound 5 (described below) was prepared according to a modified literature procedure (Du, H., et al. (2014), Organocatalytic Enantio- and Diastereoselective Conjugate Addition to Nitroolefins: When β-Ketoamides Surpass β-Ketoesters. Chem. Eur. J., 20: 8458-8466. https://doi.org/10.1002/chem.201402192). Brine solutions were prepared by dissolving the corresponding analytical grade metal sulfate or chloride salts in deionized water and adjusting final pH of the solution using NaOH. Organic phase was prepared by dissolving corresponding amounts of the extractant and co-extractant (ligand) (TOPO or Cyanex® 923) in Isopar L.

Preparation of N,N-bis(2-ethylhexyl)-3-oxobutanamide (5). A mixture of 2,2,6-trimethyl-4H-1,3-dioxin-4-one (2.84 g, 20 mmol) and N,N-bis(2-ethylhexyl)amine was heated to 90° C. and stirred overnight. The residue was purified by silica gel column chromatography (silica gel, petroleum ether/EtOAc, v/v 5:1). N,N-bis(2-ethylhexyl)-3-oxobutanamide (5) (90% yield) as a yellow oil.

Solvent Extraction Studies

General procedure: A 3-5 mL of aqueous phase consisting of ca 1.0-1900 ppm if Li⁺, 1.0-50000 ppm of Na⁺, and 1.0-20000 ppm of K⁺ as either chloride or sulfate salts at pH values between 2 and 14 was contacted with an equal volume of organic phase containing 0.1-0.8 M extractant ligands 1-5 in organic solvents. The two phases were contacted at a 1:1 up to 1:10 ratio of organic/aqueous by end-over-end rotation in individual 15 mL capacity snap-top tubes using a rotating wheel in an air box set at 25.5° C.±0.5° C. Contacts were performed in triplicate with a contact time of 1 hour. Following contacting, the triplicate samples were subjected to centrifugation at 4000 rpm for four minutes at 20° C. to separate the phases. The organic phase was then separated and stripped using 0.1-3 M HCl or H₂SO₄ respectively with a variable ratio 1:1 to 10:1 of organic/aqueous by end-over-end rotation in individual 15 mL capacity snap-top tubes using a rotating wheel in an air box set at 25.5° C.±0.5° C. Each triplicate was then sub-sampled, with 0.5-2 mL aliquots of the aqueous phases transferred to individual polypropylene tubes containing 2.5-8.0 mL of 4% HNO₃ for analysis using ICP-OES. Two samples of the initial brine were also prepared for the analysis, 500 μL were transferred to individual polypropylene tubes containing 2.5 mL of 4% HNO³. The areas found under the observed peaks were used for determining distribution (D) values of Li⁺, Na⁺ and K⁺ respectively. The concentrations of Li⁺, Na⁺ and K⁺ in the organic phase can be calculated based on the mass balance. Concentration of metals was determined by inductively coupled plasma optical emission spectroscopy (ICP-OES).

Extraction Procedure (1:1 v/v). 2.5 mL of brine solution (pH=14) was contacted with 2.50 mL of organic phase containing Cyanex® 923 and compound 5 (EHBA) in Isopar L (200 mM each). The two phases were contacted using end-over-end rotation in individual polypropylene tubes using a rotating wheel in an air box set at 25.5° C.±0.5° C. Contacts were performed in triplicate with a contact time of 1 hour 15 minutes. The samples rested on the benchtop for 10 minutes then centrifuged at 4,000 rpm for 4 minutes. The organic phase (2.0 mL) was removed and added to new polypropylene tubes containing 2.0 mL of 1.0 M H₂SO₄. The samples were contacted (1:1 v/v) using end-over-end rotation for 30 minutes.

Extraction Procedure (3:1 v/v). 0.83 mL of brine solution (pH=14) was contacted with 2.5 mL of organic phase containing Cyanex® 923 and compound 5 (EHBA) in Isopar L (200 mM each). The two phases were contacted using end-over-end rotation in individual polypropylene tubes using a rotating wheel in an air box set at 25.5° C.±0.5° C. Contacts were performed in triplicate with a contact time of 3 minutes. The samples rested on the benchtop for 10 minutes then centrifuged at 4,000 rpm for 4 minutes. The organic phase (2.0 mL) was removed and added to new polypropylene tubes containing 2.0 mL of 1.0 M H₂SO₄. The samples were contacted (1:1 v/v) using end-over-end rotation for 30 minutes.

Sample Preparation. Aliquots of the aqueous phase from extraction and stripping experiments (500 μL) were transferred to individual polypropylene tubes containing 4.5 mL of 4% HNO₃ for analysis. Solutions of the stripping solution (1.0 M H₂SO₄) and brine solution were also prepared (500 μL in 4% H₂SO₄), and all samples were analyzed using ICP-OES.

The distribution ratio (D) was measured by measuring the concentration of the metal ion in the aqueous phase after extraction and by comparing it to the initial concentration. As such, D values were determined using the following equation (Eq. 1):

$$D = \frac{{C}_{i} - {C}_{f}}{{C}_{f}} \times \frac{{V}_{a q}}{{V}_{o r g}} \qquad Eq . 1$$

where Cᵢ and C_{f} are the concentrations of the metal ions in the aqueous phase before (I=initial) and after (f=final) extraction, respectively. V_{aq} and V_{org} are the volumes of the aqueous and organic phase, respectively.
The extraction efficiency (% E) was determined by using the following equation (Eq. 2):

$$\% E = \frac{D}{( D + \frac{{V}_{a q}}{{V}_{o r g}} )} \times 100 \qquad Eq . 2$$

The percent recovery of the metals from stripping solutions can be determined using the following equation (Eq. 3):

$$\% R = \frac{{C}_{strip} - {C}_{H 2 SO 4}}{{C}_{i} \times \frac{{V}_{a q}}{{V}_{o r g}}} \times 100 \qquad Eq . 3$$

Where Cₛₜᵣᵢₚ and C_{H2SO4} is the concentration of the metal ions in the aqueous phase after and before a strip cycle, respectively.

TABLE 1
Composition of preferred brine.

| Entry | Li (ppm) | Na (ppm) | K (ppm) |
|---|---|---|---|
| 1 | 2,072.4 ± 41.5 | 57,403.9 ± 12.3 | 21,188.9 ± 92.5 |

Composition of the brine used in the extraction studies is seen in Table 1. Generally, the amount of lithium is order of magnitude lower than the amount of other alkali metals such as sodium and potassium. In many instances, both chloride and sulfate brines were prepared of similar composition and concentration for the extraction studies. The pH of the brine was regulated by the addition of NaOH solution.

TABLE 2
Extraction performance of the variable phase ratio of extractants 1-3

| Entry | | SF_{Li/Na} | SF_{Li/K} | % E_{Li} | % E_{Na} | % E_{K} |
|---|---|---|---|---|---|---|
| 1 | 1:1 O:A1 | >10000 | >10000 | 15.0 | 0 | 0 |
| 2 | 3:1 O:A1 | >10000 | >10000 | 23.2 | 0 | 0 |
| 3 | 1:1 O:A2 | >10000 | 8948 | 14.8 | 0 | 0.002 |
| 4 | 3:1 O:A2 | >10000 | >10000 | 23.4 | 0 | 0 |
| 5 | 1:1 O:A3 | 1363 | >10000 | 13.2 | 0.01 | 0 |
| 6 | 3:1 O:A3 | >10000 | >10000 | 17.3 | 0 | 0 |

As can be clearly seen in Table 2, a variety of extractants have high affinity for lithium over sodium and potassium. Specifically, at low organic to aqueous ratio (entry 1, 3 and 5), the molar equivalency of extractant is insufficient to achieve full extraction of lithium form the brine. This may occur by insufficient concentration of the extractant in the organic phase relative to the presence of cations, specifically lithium, in the aqueous phase. The separation factors for both Na and K are in excess of 100 in all cases. Next, by increasing the organic to aqueous phase ratio and performing the extraction studies (entry 2, 4 and 6) it is evident that the total amount of lithium that is extracted into the organic phase is also increasing. The amount of the extractant was increased relative to the amount of lithium present in the aqueous solution; therefore, the amount of lithium extracted into the organic phase was also increased. Notably, all three extractants 1-3 have a high affinity and selectivity towards lithium over sodium and potassium ions and are very effective at discriminating and sequestering lithium even at high concentrations of interfering ions. To further probe the effect of phase ratio and concentration on the extraction ability and selectivity, extraction studies were performed using extractant 2 and TOPO as a co-extractant in Isopar L diluent. The results of these experiments are presented in Table 3.

TABLE 3
Percent metal extraction using extractant 2 solution

| Entry | | % E_{Li} | % E_{Na} | % E_{K} |
|---|---|---|---|---|
| 1 | 1:1 O:A 0.1M 2 | 35.8 | 0.0 | 0.2 |
| 2 | 2:1 O:A 0.1M 2 | 72.6 | 0.0 | 0.5 |
| 3 | 3:1 O:A 0.1M 2 | 100.0 | 8.2 | 0.9 |
| 4 | 1:1 O:A 0.2M 2 | 65.9 | 1.0 | 0.1 |
| 5 | 2:1 O:A 0.2M 2 | 100.0 | 19.6 | 0.5 |
| 6 | 3:1 O:A 0.2M 2 | 100.0 | 17.5 | 0.7 |

From Table 3 it is clearly demonstrated that extractant 2 has a very high affinity for lithium in the presence of sodium and potassium, especially when the amount of the ligand as expressed in the concentration and volume ratio, is equal or lower to the total amount of lithium present in the aqueous phase (Entry 1, 2 and 4). However, when the amount of extractant becomes super stoichiometric with respect to lithium in the aqueous phase, some amount of sodium gets extracted as well (Entry 3, 5 and 6). This may be explained by the mechanism of action of the extraction process, i.e. deprotonation-complexation. When the total amount of extractant 2 in the organic phase is larger or equal to the total amount of lithium present in the aqueous phase, all of the extractant can bind with the lithium ions. This in turn results in high lithium extraction and low sodium and potassium extraction (Entry 1, 2 and 4). Whenever the total amount of extractant is lower than the total amount of lithium in the aqueous phase, in addition to extracting all of the lithium, some amount of sodium and to a lesser degree potassium may get extracted into the organic phase (Entry 3, 5 and 6). Next, the performance of relatively more acidic extractant 2 to extractant 4 was compared under variable concentration and organic to aqueous phase ratio to elucidate their selectivity and lithium extraction profiles.

Table 4 (below) shows the performance of extractants 2 and 4 under a dynamic extraction range, BDL stands for “below the detection limit” of the instrument. Specifically, concentration of the extractant 2 is 0.2 M and extractant 4 is 0.1 M. Based on the stoichiometry and concentration of lithium, total amount of the extractant 2 and 4 are equal when O:A ratio is 2:1 and 4:1, respectively (Entry 1 and 5). Extractant 2 is competent at extracting all of the lithium from the brine under experimental conditions (Entry 1) in addition to significant amount of sodium. Extractant 4 on the other hand is also a very selective lithium extractant, providing especially clean extraction of lithium when the total amount of the extractant is lower than the total amount of lithium available in the aqueous phase (Entry 2 and 3). When the amount the extractant 4 becomes super stoichiometric with respect to lithium, a small amount of sodium gets extracted as well (Entry 4 and 5). It is worth noting that extractant 2 and extractant 4 show high selectivity and affinity toward lithium; however, extractant 4 is more selective under the experimental conditions since it extracts less sodium compared to extractant 2 (Entry 1 and 5). Next, additional extraction experiments were performed to verify the efficiency of metal extraction and metal stripping from the loaded organic phase.

TABLE 4
Metal concentration in organic phase using extractants 2 and 4

| Entry | | Li (ppm) | Na (ppm) | K (ppm) |
|---|---|---|---|---|
| 1 | 2:1 O:A 2 | 1934 | 2237 | 22 |
| 2 | 1:1 O:A 4 | 615 | BDL | BDL |
| 3 | 2:1 O:A 4 | 1275 | BDL | BDL |
| 4 | 3:1 O:A 4 | 1753 | 136 | BDL |
| 5 | 4:1 O:A 4 | 1935 | 486 | BDL |

In Table 5 (below), varying concentration and phase ratios were used to estimate the extraction performance and stripping efficiency of the loaded organic phase. Brine composition used for these specific extraction experiments can be seen in Entry 1. Both extractant 2 and 4 show similar lithium extraction performance when adjusted for relative amounts (Entry 2 and 6). The overall selectivity and extraction ability (Entries 2-6) trends of extractants 2 and 4 are similar to those observed in Table 4. Entries 7-11 clearly indicate that the vast majority of the metal extracted into the organic phase is efficiently stripped with a single acid contact. The overall amount of all the metal ions after the 1^{st} acid stripping remaining in the organic phase was below the detection limit of the instrument, as indicated by the results of the second stripping cycle. These results indicate that the extraction and strip cycle is highly efficient and does not require more than a single metal stripping stage. Next, extraction-concentration experiments were performed to afford markedly higher lithium concentration of the lithium compared to the initial concentration of lithium in the brine by manipulating the organic to aqueous phase ratio and stripping with 2 M H₂SO₄.

TABLE 5
Metal concentration after solvent extraction and
the 1^{st} and 2^{nd} stripping using H₂SO₄ solution.

| Entry | | Li (ppm) | Na (ppm) | K (ppm) |
|---|---|---|---|---|
| 1 | Brine composition | 1850 | 46000 | 19500 |
| 2 | 2:1 O:A 2 | 1826 | 1409 | 36 |
| 3 | 1:1 O:A 4 | 623 | 8 | BDL |
| 4 | 2:1 O:A 4 | 1314 | 23 | BDL |
| 5 | 3:1 O:A 4 | 1757 | 100 | BDL |
| 6 | 4:1 O:A 4 | 1843 | 310 | BDL |
| 7 | 2:1 O:A 2 2^{nd} strip | BDL | BDL | BDL |
| 8 | 1:1 O:A 4 2^{nd} strip | BDL | BDL | BDL |
| 9 | 2:1 O:A 4 2^{nd} strip | BDL | BDL | BDL |
| 10 | 3:1 O:A 4 2^{nd} strip | BDL | BDL | BDL |
| 11 | 4:1 O:A 4 2^{nd} strip | BDL | BDL | BDL |

Table 6 (below) summarizes the results of the extraction-metal concentration sequence that is achieved by judicious manipulation of the organic to aqueous phase ratio. Entry 5 shows initial lithium concentration in the brine expressed in parts per million (ppm) and entry 6 shows the same initial concentration expressed in mol/L (M) concentration. After contacting the organic phase containing 0.8 M of Compound 4 and 0.8 M Cyanex® 923 with an O:A phase ratio of 1:4, loaded organic was separated and further stripped with 2.0 M H₂SO₄, and contacted with an O:A phase ratio of 1:4. The resulting concentration of lithium expressed in various ways are seen in Entries 1-4, with the overall concentration factor around 17 (Entry 7). By extracting lithium into a very concentrated solution of the extractant 4 in the organic diluent, the overall concentration of lithium in the organic phase was higher than the one in the initial aqueous solution. Specifically, in a limiting case, when the amount of lithium in the aqueous solution is exceeding or a least equal to the total amount of the organic extractant, theoretical lithium concentration in the organic phase will be approaching that of the extractant. Subsequently, by manipulating the organic to aqueous phase ratio and the acid concentration during the stripping stage, additional concentration of the lithium is accomplished, effectively by further decreasing the volume of the solution. In this case, 2 M sulfuric acid can provide twice the amount of protons available for the stripping. Therefore, in the limiting case, lithium concentration can be achieved approaching that of the acid in the form of Li₂SO₄.

TABLE 6
Concentration and composition of aqueous
strip solutions of extractant 0.8 M of Compound 4
(DIPI) with 0.8 M Cyanex ® 923 in Isopar L after
extraction-concentration from brine using ICP-OES.
(contact time = 1 h 15 m; temperature = 25.5° C.)

| Entry | | Value |
|---|---|---|
| 1 | Li Concentration (ppm) after extraction | 23,990 |
| 2 | Li Concentration after extraction (M) | 3.872 |
| 3 | Li Concentration after extraction (wt %) | 2.399 |
| 4 | Li₂SO₄ Concentration after extraction (M) | 1.936 |
| 5 | Initial Li concentration before extraction (ppm) | 1367 |
| 6 | Initial Li concentration before extraction (M) | 0.2243 |
| 7 | Li Concentration factor | 17 |

Table 7 (below) shows the extraction behavior of the extractant 5 in the presence of co-extractant Cyanex® 923 with the organic to aqueous phase ratio 3:1. The amount of lithium extracted under these conditions is approaching 100% and the distribution coefficient value and percentage of sodium and potassium extracted is very low. This result may also be attributed to a much lower acidity of the extractant 5 compared to extractants 1-4 (Tables 2-5).

TABLE 7
Analysis of aqueous solutions of extractant 5 with Cyanex ® 923 in
Isopar L after extraction from brine using ICP-OES. (contact time =
1 h 15 m; temperature = 25.5° C.)

| Entry | V_{org}/V_{aq} | brine pH | D_{Li} | % E_{Li} | D_{Na} | % E_{Na} | D_{K} | % E_{K} |
|---|---|---|---|---|---|---|---|---|
| 1 | 3:1 O:A5 | pH = 14 | 17.1 | 98.1 | 0.01 | 1.8 | 0.01 | 1.3 |

Table 8 (below) shows the extraction behavior of the extractant 5 under two different sets of organic to aqueous (O:A) phase ratios. In both cases (Entry 1 and 2), the recovery of lithium is very high and approaching 100%. Notably, in both cases, the amount of sodium and potassium is very low and stays consistent, which demonstrates very high selectivity toward lithium.

TABLE 8
Analysis of aqueous strip solutions of extractant 5 with Cyanex ®
923 in Isopar L after extraction from brine using ICP-OES. (contact
time = 1 h 15 m; temperature = 25.5° C.)

| | | brine | | % | Na | % | K | % |
|---|---|---|---|---|---|---|---|---|
| Entry | V_{org}/V_{aq} | pH | Li (ppm) | R_{Li} | (ppm) | R_{Na} | (ppm) | R_{K} |
| 1 | 1:1 O:A2 | pH = | 1,470 ± | 98.1 | 83.00 ± | <1 | 18.0 ± | <1 |
| | | 14 | 83.2 | | 5.7 | | 1.5 | |
| 2 | 3:1 O:A2 | pH = | 533.5 ± | 99.5 | 91.9 ± | <1 | 17.5 ± | <1 |
| | | 14 | 7.0 | | 1.7 | | 0.8 | |

While there have been shown and described what are at present considered the preferred embodiments of the invention, those skilled in the art may make various changes and modifications which remain within the scope of the invention defined by the appended claims.

What is claimed is:

1. A lithium extractant compound having the following structure: 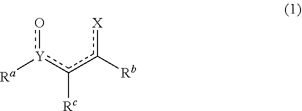
wherein:
R^{a} and R^{b} are independently selected from the group consisting of hydrocarbon groups (R), —OR, —NRR′, —SR, —SO₂R, —SO₂NR₂, —C(O)R, —C(O)OR, —C(O)NRR′, —C(S)OR, —C(O)SR, and —C(S)NRR′;
R^{c} is selected from R′ groups;
wherein the hydrocarbon groups (R) independently contain 1-30 carbon atoms and are selected from alkyl groups, alkenyl groups, alkynyl groups, cycloalkyl groups, cycloalkenyl groups, aromatic groups, and heteroaromatic groups, any of which are optionally substituted with fluorine, and wherein said cycloalkyl groups, cycloalkenyl groups, aromatic groups, and heteroaromatic groups are further optionally substituted with one or more groups selected from the group consisting of alkyl (R″) groups containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂;
R′ is selected from H and R groups;
X is O or OH;
Y is C or N, wherein, when Y is N, then R^{a} is R;
the dotted lines indicate optional presence of carbon-carbon double bonds;
R^{a} and R^{c} optionally interconnect via their R groups to form ring A, wherein ring A is optionally substituted with one or more groups selected from the group consisting of alkyl (R″) groups containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂;
R^{b} and R^{c} optionally interconnect via their R groups to form ring B, wherein ring B is optionally substituted with one or more groups selected from the group consisting of alkyl (R″) groups containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂; and
R^{a}, R^{b}, and R^{c} optionally interconnect via their R groups to form a fused bicyclic ring system, wherein the fused bicyclic ring system is optionally substituted with one or more groups selected from the group consisting of alkyl (R″) groups containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂;
provided that R^{a} and R^{b} are not both alkyl groups, and if R^{a} is an alkyl group and R^{b} is an aromatic group, or if R^{a} and R^{b} are the same aromatic groups, then at least one of the aromatic groups is substituted with a group selected from the group consisting of alkyl (R″) groups containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂.

2. The lithium extractant compound of claim 1, wherein the lithium extractant compound has the following structure: 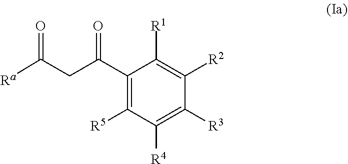
wherein R^{a} is as defined in claim 1, and R¹, R², R³, R⁴, and R⁵ are independently selected from the group consisting of H, alkyl (R″) groups containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂.

3. The lithium extractant compound of claim 2, wherein at least one of R¹, R², R³, R⁴, and R⁵ is an —OR″ group.

4. The lithium extractant compound of claim 2, wherein the lithium extractant compound has the following structure: 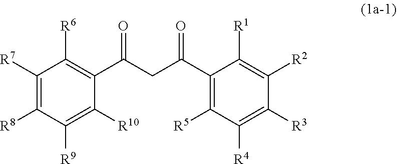
wherein R¹, R², R³, R⁴, R⁵, R⁶, R⁷, R⁸, R⁹, and R¹⁰ are independently selected from the group consisting of alkyl (R″) groups containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂.

5. The lithium extractant compound of claim 2, wherein R^{a} is an alkyl or alkenyl group containing 2-30 carbon atoms.

6. The lithium extractant compound of claim 1, wherein the lithium extractant compound has the following structure: 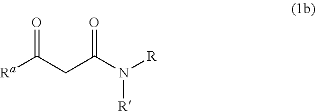
wherein R^{a}, R, and R′ are as defined in claim 1.

7. The lithium extractant compound of claim 6, wherein R^{a} is an aromatic group optionally substituted with one or more groups selected from the group consisting of alkyl (R″) groups containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂.

8. The lithium extractant compound of claim 1, wherein the lithium extractant compound has the following structure: 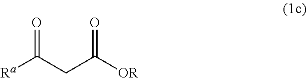
wherein R^{a} and R are as defined in claim 1.

9. The lithium extractant compound of claim 7, wherein R^{a} is an aromatic group optionally substituted with one or more groups selected from the group consisting of alkyl (R″) groups containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂.

10. The lithium extractant compound of claim 1, wherein the lithium extractant compound has the following structure: 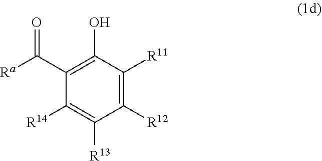
wherein:
R^{a} is as defined in claim 1; and
R¹¹, R¹², R¹³, and R¹⁴ are independently selected from the group consisting of H, alkyl (R″) groups containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂.

11. The lithium extractant compound of claim 10, wherein R^{a} is an aromatic group optionally substituted with one or more groups selected from the group consisting of alkyl (R″) groups containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂.

12. The lithium extractant compound of claim 1, wherein the lithium extractant compound has the following structure: 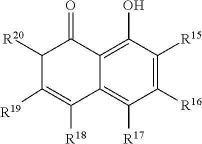
wherein R¹⁵, R¹⁶, R¹⁷, R¹⁸, R¹⁹, and R²⁰ are independently selected from the group consisting of H, alkyl (R″) groups containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂, provided that at least one of R¹⁵, R¹⁶, R¹⁷, R¹⁸, R¹⁹, and R²⁰ is selected from the group consisting of R″, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂.

13. The lithium extractant compound of claim 1, wherein the lithium extractant compound has the following structure: 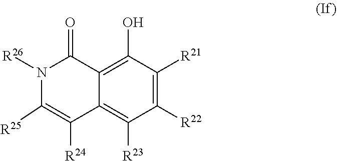
wherein:
R²¹, R²², R²³, R²⁴, and R²⁵ are independently selected from the group consisting of H, alkyl (R″) groups containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂; and
R²⁶ is selected from the group consisting of R″, —NHR″, —NR″₂, —NO₂, —C(O)R″, —C(O)OR″, —C(O)NHR″, —C(O)NR″₂, —C(S)OR″, —C(O)SR″, —C(S)NHR″, and —C(S)NR″₂.

14. The lithium extractant compound of claim 13, wherein at least one of R²¹, R²², R²³, R²⁴, and R²⁵ is selected from the group consisting of R″, —OR″, —NHR″, —NR″₂, —NO₂, —SR″, —SO₂R″, —SO₂NR″₂, —C(O)R″, —C(O)OR″, —C(O)NHR″, —C(O)NR″₂, —C(S)OR″, —C(O)SR″, —C(S)NHR″, and —C(S)NR″₂.

15. The lithium extractant compound of claim 1, wherein the lithium extractant compound has the following structure: 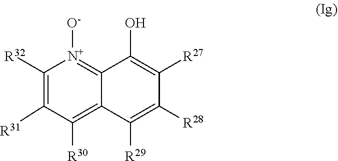
wherein R²⁷, R²⁸, R²⁹, R³⁰, R³¹, and R³² are independently selected from the group consisting of H, alkyl (R″) groups containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂, provided that at least one of R²⁷, R²⁸, R²⁹, R³⁰, R³¹, and R³² is selected from the group consisting of R″, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂.

16. The lithium extractant compound of claim 1, wherein at least one of R^{a} and R^{b} is selected from the group consisting of —C(O)R, —C(O)OR, —C(O)NRR′, —C(S)OR, —C(O)SR, —C(S)NRR′, and —NRR′.

17. The lithium extractant compound of claim 1, wherein R^{a} and R^{b} are independently selected from the group consisting of —C(O)R, —C(O)OR, —C(O)NRR′, —C(S)OR, —C(O)SR, —C(S)NRR′, and —NRR′.

18. An aqueous-insoluble hydrophobic solution useful for extracting lithium from an aqueous solution, comprising a chelating lithium extractant compound dissolved in an aqueous-insoluble hydrophobic solvent, wherein the lithium extractant compound has the following structure: 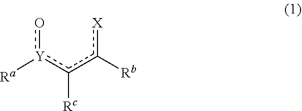
wherein:
R^{a} and R^{b} are independently selected from the group consisting of hydrocarbon groups (R), —OR, —NRR′, —SR, —SO₂R, —SO₂NR₂, —C(O)R, —C(O)OR, —C(O)NRR′, —C(S)OR, —C(O)SR, and —C(S)NRR′;
R^{c} is selected from R′ groups;
wherein the hydrocarbon groups (R) independently contain 1-30 carbon atoms and are selected from alkyl groups, alkenyl groups, alkynyl groups, cycloalkyl groups, cycloalkenyl groups, aromatic groups, and heteroaromatic groups, any of which are optionally substituted with fluorine, and wherein said cycloalkyl groups, cycloalkenyl groups, aromatic groups, and heteroaromatic groups are further optionally substituted with one or more groups selected from the group consisting of alkyl (R″) groups containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂;
R′ is selected from H and R groups;
X is O or OH;
Y is C or N, wherein, when Y is N, then R^{a} is R;
the dotted lines indicate optional presence of carbon-carbon double bonds;
R^{a} and R^{c} optionally interconnect via their R groups to form ring A, wherein ring A is optionally substituted with one or more groups selected from the group consisting of alkyl (R″) groups containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂;
R^{b} and R^{c} optionally interconnect via their R groups to form ring B, wherein ring B is optionally substituted with one or more groups selected from the group consisting of alkyl (R″) groups containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂; and
R^{a}, R^{b}, and R^{c} optionally interconnect via their R groups to form a fused bicyclic ring system, wherein the fused bicyclic ring system is optionally substituted with one or more groups selected from the group consisting of alkyl (R″) groups containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂;
provided that R^{a} and R^{b} are not both alkyl groups, and if R^{a} is an alkyl group and R^{b} is an aromatic group, or if R^{a} and R^{b} are the same aromatic groups, then at least one of the aromatic groups is substituted with a group selected from the group consisting of alkyl (R″) groups containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂.

19. The liquid solution of claim 18, wherein the aqueous-insoluble hydrophobic solution further comprises a non-chelating co-extractant molecule dissolved in the aqueous-insoluble hydrophobic solvent, wherein the non-chelating co-extractant molecule has a single donor group that can form a single coordinate bond with a lithium ion.

20. The liquid solution of claim 19, wherein the non-chelating co-extractant molecule is selected from the group consisting of R′C(O)NR′₂, R′₂NC(O)NR′₂, C(O)R₂, P(O)R₃, S(O)R₂, and N⁺(O⁻)R′₃, wherein:
R is independently selected from hydrocarbon groups containing 1-30 carbon atoms and selected from alkyl groups, alkenyl groups, alkynyl groups, cycloalkyl groups, cycloalkenyl groups, and aromatic groups, with no heteroatom substitution;
R′ is selected from H and R; and
one or more R groups in P(O)R₃ and C(O)R₂ can be replaced with an OR group.

21. A method for extracting lithium from aqueous solution, the method comprising:
(i) providing an aqueous solution containing lithium and having a pH sufficient to deprotonate a chelating lithium extractant compound according to Formula (1);
(ii) contacting the aqueous solution with an aqueous-insoluble hydrophobic solution comprising the chelating lithium extractant compound dissolved in an aqueous-insoluble hydrophobic solvent to result in deprotonation of the chelating lithium extractant compound, chelation of lithium in the aqueous solution by the deprotonated form of the chelating lithium extractant compound, and resultant extraction of the lithium from the aqueous solution into the aqueous-insoluble hydrophobic solution, wherein the chelating lithium extractant compound has the following structure: 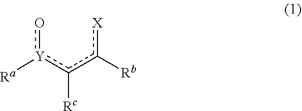
wherein:
R^{a} and R^{b} are independently selected from the group consisting of hydrocarbon groups (R), —OR, —NRR′, —SR, —SO₂R, —SO₂NR₂, —C(O)R, —C(O)OR, —C(O)NRR′, —C(S)OR, —C(O)SR, and —C(S)NRR′;
R^{c} is selected from R′ groups;
wherein the hydrocarbon groups (R) independently contain 1-30 carbon atoms and are selected from alkyl groups, alkenyl groups, alkynyl groups, cycloalkyl groups, cycloalkenyl groups, aromatic groups, and heteroaromatic groups, any of which are optionally substituted with fluorine, and wherein said cycloalkyl groups, cycloalkenyl groups, aromatic groups, and heteroaromatic groups are further optionally substituted with one or more groups selected from the group consisting of alkyl (R″) groups containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂;
R′ is selected from H and R groups;
X is O or OH;
Y is C or N, wherein, when Y is N, then R^{a} is R;
the dotted lines indicate optional presence of carbon-carbon double bonds;
R^{a} and R^{c} optionally interconnect via their R groups to form ring A, wherein ring A is optionally substituted with one or more groups selected from the group consisting of alkyl (R″) groups containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂;
R^{b} and R^{c} optionally interconnect via their R groups to form ring B, wherein ring B is optionally substituted with one or more groups selected from the group consisting of alkyl (R″) groups containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂; and
R^{a}, R^{b}, and R^{c} optionally interconnect via their R groups to form a fused bicyclic ring system, wherein the fused bicyclic ring system is optionally substituted with one or more groups selected from the group consisting of alkyl (R″) groups containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂;
provided that R^{a} and R^{b} are not both alkyl groups, and if R^{a} is an alkyl group and R^{b} is an aromatic group, or if R^{a} and R^{b} are the same aromatic groups, then at least one of the aromatic groups is substituted with a group selected from the group consisting of alkyl (R″) groups containing 1-20 carbon atoms, —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂.

22. The method of claim 21, wherein said method further comprises:
(iii) stripping lithium from the aqueous-insoluble hydrophobic solution by contacting the aqueous-insoluble hydrophobic solution with an aqueous stripping solution having a pH sufficient to result in reprotonation of the chelating lithium extractant compound and simultaneous release of the lithium from the aqueous-insoluble hydrophobic solution into the aqueous stripping solution.

23. The method of claim 21, wherein the aqueous-insoluble hydrophobic solution further comprises a non-chelating co-extractant molecule dissolved in the aqueous-insoluble hydrophobic solvent, wherein the non-chelating co-extractant molecule has a single donor group that can form a single coordinate bond with a lithium ion.

24. The method of claim 23, wherein the non-chelating co-extractant molecule is selected from the group consisting of R′C(O)NR′₂, R′₂NC(O)NR′₂, C(O)R₂, P(O)R₃, S(O)R₂, and N⁺(O⁻)R′₃, wherein:
R is independently selected from hydrocarbon group containing 1-30 carbon atoms and selected from alkyl groups, alkenyl groups, alkynyl groups, cycloalkyl groups, cycloalkenyl groups, and aromatic groups, with no heteroatom substitution;
R′ is selected from H and R; and
one or more R groups in P(O)R₃ and C(O)R₂ can be replaced with an OR group.
